(19) United States
(12) Patent Application Publication
(10) Pub. No.: US 2005/0090786 A1
(43) Pub. Date: Apr. 28, 2005

(54) Dual flex insertion device for enema, douche or colonic irrigation

(76) Inventor: In E. Moon, Irvine, CA (US)

Correspondence Address: SHERMAN & SHALLOWAY, 413 North Washington Street, Alexandria, VA, 22314, US

(21) Appl. No.: 10/690,592

(22) Filed: Oct. 23, 2003

(51) Int. Cl. A61M 31/00

(52) U.S. Cl. 604/275

(57) ABSTRACT

An insertion device and rectal tip assembly having a dual flex allowing for the safe and non-traumatic insertion and removal of tubing into a body cavity. The dual flex insertion tubing has a flexible part and a rigid base part wherein the soft flexible part provides sufficient play and support when inserting the tubing into a body cavity while the rigid part provides a supporting base for the soft flexible part of the tubing. A first tubular portion has a first flexural modulus wherein said first flexural modulus is sufficiently pliable to allow for repeated non-traumatic insertion and is in communication with a second tubular portion having a second flexural modulus sufficiently rigid to support the first tubular portion but also has a minimum bend radius that allows for sufficient play of the second tubular portion. The dual flex insertion device can be used with an enema, douche or colonic irrigation device or any other device used to introduce liquid into a body cavity. The dual flex insertion device can also be used alone or in combination with toilet attachments as described in U.S. Pat. No. 5,946,741, the disclosure of which is incorporated herein by reference.

### INTRODUCTION

The invention relates to an insertion device and more particularly to a rectal tip assembly having a dual flex. The dual flex of the rectal assembly allows for the safe and non-traumatic insertion and removal of tubing into a body cavity. In particular, the dual flex insertion tubing has a flexible part and a rigid base part wherein the soft flexible part provides sufficient play and support when inserting the tubing into a body cavity while the rigid part provides a supporting base for the soft flexible part of the tubing. The dual flex insertion device can be used with an enema, douche or colonic irrigation device or any other device used to introduce liquid into a body cavity. The dual flex insertion device can also be used alone or in combination with toilet attachments as described in U.S. Pat. No. 5,946,741, the disclosure of which is incorporated herein by reference.

### BACKGROUND OF THE INVENTION

Irrigating devices for introducing liquid into a body cavity such as the colon have been developed and used wherein a wash fluid is introduced into the colon by the force of gravity through an irrigator positioned in the rectum. After sufficient pressure has been created, the flow inwards is stopped and the pressure increased until waste fluid is allowed to flow out. However, known irrigators are complicated and prohibitively expensive as well as being unsuitable for home use due to any number of features.

Foremost of these is the requirement found in many known irrigators that a conduit remains positioned inside the body cavity during the entire treatment period. This complicates the design of the irrigation device because many considerations such as size, sealing properties and unwanted leakage must be accommodated in order for the device to function properly with the requisite efficiency and safety. For example, in order to prevent contamination of the device from outgoing waste fluid, known devices employ a conduit having an open ended nozzle with a tapered inner end containing a removable rounded nose obturator. These types of irrigators define a conduit leading into the open ended nozzle wherein the obturator is placed inside the nozzle which supplies both the incoming clean fluid and the outgoing waste fluid. The notable feature of these types of devices is that they are not removed during the irrigation process. Although this minimizes the need for sterilizing the irrigator after it has been used or prior to subsequent use, these types of devices are typically very complicated and require a skilled operator and are not appropriate for home use. Furthermore, various and complicated sealing and anchoring mechanisms are required in order to maintain a proper seal with an inner sphincter wall.

One way to overcome this problem is by irrigating the colon through a series of irrigations instead of introducing a single conduit into the rectum that is not removed during the procedure. The series of irrigation has the same effect of saturating the colon which allows for the easy removal of any plaster-like fecal matter. In particular, the fecal coating in the colon must be thoroughly soaked and saturated with water so that removal can take place gradually and comfortably. Saturation of the colon is critical to removing the fecal matter because removing fecal matter too rapidly would cause the inner lining of the colon to become raw and cause discomfort and pain to the patient.

This gradual removal can be accomplished by introducing several ounces of water at a time to saturate the colon wherein water is subsequently expelled after each introduction. The process is repeated until the colon is sufficiently saturated to provide for the easy removal of the plaster-like fecal coating. However, such an apparatus must be inserted and removed any number of times, thereby increasing the likelihood of damage to the rectum as well as the surrounding tissues. For example, if the irrigating tube is inserted beyond a determined safe point, the tube could cause colon perforation, sepsis and severe injury to the anal canal. On the other hand, if the irrigating tube is not inserted far enough into the body cavity, water does not satisfactorily saturate the desired length of the colon. The difficulty of producing a suitable irrigating tube is further complicated by the natural curvature of the human colon.

Setting a safe point beyond which an irrigating tube should not be inserted may overcome this obstacle. One such safe point is generally defined as the point where the sigmoid colon forms a loop in the rectum. Although the position and shape of the sigmoid colon depends on (1) length of the colon, (2) length of mesocolon, (3) condition of distension and (4) condition of the rectum and bladder, the average length can be estimated from the inlet of the lower pelvis down the descending colon. Accordingly, a safe point can be estimated to avoid misdirected or improper introduction of an irrigating tube into the colon. However, the depth of the insertion is not the only source of injury.

Known insertion devices have been and continue to be constructed of a hard injection molded plastic formed into a catheter or lumen of a generally cylindrical nature which may not only be ineffective or harmful but oftentimes accidentally puncture or damage the colon. Despite the danger of possible injury, the lumen or catheter of known devices continue to be uniformly constructed and fail to allow for any flexural variation between distal and proximal ends. While these types of known insertion devices continue to have utility, they nevertheless remain inherently dangerous due to the rigid aspect of the hard injection molded plastic.

The danger is so great that many manufacturers warn that tips should not be inserted into the colon more than three inches past the anus. Manufacturers further warn that use of their devices may cause excessive pressure against the bowel wall leading to irritation and even possibly to severe and debilitating mechanical problems. To avoid this possibility, known insertion devices include many complicated warnings and sometimes even state the need for extensive training prior to their use.

One way to reduce the possibility of injury is by providing an extremely pliable insertion tip. The extreme softness or flexibility of the material prevents injury to the rectum during the repeated insertion and removal of the catheter and further reduces the likelihood of injury. However, the extreme pliability of the insertion tip may prevent repeated insertion and removal of an insertion device and sometimes even prevents insertion of the tip into the anus. This lack of support forces the patient to lie horizontally on a sideboard in the supine position in order to receive treatment, which requires the use of a bulky sideboard adjacent to the waste receiving receptacle. While the use of a sideboard may be possible in a clinical setting it is practicably impossible in a home setting where colonic irrigators are most commonly used in conjunction with regular toilet fixtures. The size and placement of a horizontal sideboard would prevent its use in most residential bathrooms. Further, the volume of water involved makes it difficult to perform colonic irrigation while remaining clean.

Clearly, there is a need for an economical and safe way to use a colonic device, which does not require the use of a sideboard or any number of other complicated devices. There is also a need for providing home colonics that allow for the repeated insertion and removal of an insertion tip that does not cause injury to the colon or surrounding tissues. Still further, and particularly due to the complexity and danger of possible injury through unsupervised use of known irrigators, there is a need for an economical and safe insertion device that can easily be used in conjunction with toilet attachments. The apparatus should be easily and repeatedly insertable into the rectum without injury of any kind and be fabricated in manner convenient for home use or in conjunction with other known toilet attachments. The apparatus must also be easy to use and not require extensive and complicated training for its use. It is to such a device along with various embodiments that the present invention is directed.

### SUMMARY OF THE INVENTION

In accordance with the present invention, one aspect of the invention contemplates a simple yet functional dual flex irrigator that progressively irrigates and saturates the colon after insertion, removal and reinsertion into the rectum. The dual flex irrigator of the invention has a dual flex assembly for delivering liquid into a body cavity which has a first soft tubular portion allowing for non-traumatic insertion of the first tubular portion into a body cavity and a second tubular portion removably attached to the first tubular portion having a second flex that provides a rigid base for the first soft tubular portion. The dual flex assembly device can be inserted into the rectum repeatedly without injury and has two separate flexions wherein the first flexion allows for the safe and repeated insertion and removal of a lumen or catheter into the rectum and the second flexion provides for a rigid base that supports the first soft tubular portion.

The dual flex assembly device can also have a first tubular portion having a proximal end, a distal functional end, and a first flexural modulus wherein said first flexural modulus being sufficiently pliable to allow for repeated non-traumatic insertion and removal of the first tubular portion into and from the rectum. The proximal end of the first tubular portion should be in communication with a second tubular portion which also has a proximal end, a distal end, and a second flexural modulus. Here, the second flexural modulus is sufficiently rigid to support the first tubular portion but yet allows for a minimum bend radius that provides sufficient play to the second tubular portion wherein it preferentially allows for insertion of the distal end of the first tubular portion into the rectum.

Another aspect of the invention includes an obturator wherein said obturator is comprised of a bulbous end and a second end separated by a shaft, said bulbous end being oriented with the distal end of said first tubular portion and being adapted to fit into a patient's rectum wherein said shaft has a third flexural modulus which is at least as flexible as said first flexural modulus of said first tubular part and is further adapted to be received within both first and second tubular portions. The obturator can have a removable rounded nose obturator extending from the distal end of the first tubular portion with the body of the round nose obturator bearing against the inner surface of the distal end of the first tubular portion. The obturator is removable through the dual flex assembly in order for the fluid supply to enter the irrigator. Notably, a conduit supply of fluid is preferably offset in relation to the longitudinal axis of the dual flex assembly nozzle to provide a swirling action to the entering fluid. The nozzle has an outwardly extending circumferential flange intermediate of the inner end, which limits the insertion of the irrigator. A conduit attached to the other end of the nozzle has variable control means thereon to control the pressure of fluid in the nozzle and its rate of discharge from said nozzle.

Still another aspect of the invention involves a toilet attachment in communication with the dual flex assembly wherein the toilet attachment can be positioned between a toilet seat and a toilet bowl. The dual flex assembly can be removably attached or welded to the toilet attachment and be used in place of known insertion tips. The toilet attachment itself comprises a tubular irrigation assembly and a base plate wherein the base plate has an upper and lower surface, a rear end and a front end. When installed, the rear end faces a rear portion of the bowl and the front end faces a front portion of the bowl. The base plate has ports to receive and secure the fluid line and the irrigation assembly. At least one of these ports is located at the front end. The toilet attachment can easily be attached to and removed from the toilet to store the toilet attachment when not in use. The irrigation assembly and the fluid line can be removed from the base plate, which allows for easy cleaning and replacement of parts that get into contact with a user's body and/or a user's fluids or excrements.

Still yet another aspect of the invention involves a toilet attachment wherein the dual flex irrigation assembly has a means for preventing insertion of the irrigation tip into a user's body cavity beyond a predetermined distance. The means is disposed on the dual flex assembly at a preset distance from the tip. The means may have a variety of shapes such as a “U” shape, a double “L” shape, a lateral “U” shape, or a lateral “S” shape. The means is sized to prevent unintentional insertion into a rectum, vagina or body cavity. The various embodiments of the safety feature further adds flexibility to the irrigation assembly to allow a user more mobility and provide for more comfort than a hard tube but is desirably formed on the second rigid part having a lesser flexural modulus.

Another aspect of the invention involves a dual flex irrigation assembly suitable for a toilet attachment. The dual flex assembly comprises a tubular body having a first and second end. The first end is open, and can be removable attached to a base plate of the toilet attachment so that fluid can be fed to the dual flex assembly. The dual flex assembly further comprises an irrigation tip and a means for limiting insertion of the tip. The tip is positioned at the second end of the tubular body, and the means is disposed on the tubular body at a preset distance from the irrigation tip for limiting insertion of the tip into a user's body to a preset maximum distance. The dual flex irrigation assembly is removably attachable to the base plate so that various users may use the same base plate and change only the irrigation assembly which is partly inserted into a body cavity.

### BREIF DESCRIPTION OF THE DRAWINGS

The invention will now be explained in greater detail by the following description and specific embodiments and with the aid of the accompanying drawings. The illustrated embodiments are intended to illustrate, but not to limit the invention. The drawings contain the following figures:

FIG. 1A is a sectional view of a dual flex rectal assembly configured in a U-shaped embodiment.

FIG. 1B is an exploded view of a dual flex rectal assembly configured in a U-shaped embodiment prior to welding.

FIG. 2A is a sectional view of a dual flex rectal assembly configured in a straight embodiment.

FIG. 2B is an exploded view of a dual flex rectal assembly configured in a straight embodiment prior to welding.

FIG. 3A is a perspective of a dual flex rectal assembly configured as a conventional enema tip.

FIG. 3B is an exploded perspective of a dual flex rectal assembly configured as a conventional enema tip.

FIG. 4A is a plane view of a dual flex rectal assembly configured to receive an obturator.

FIG. 4B is an exploded plane view of a dual flex rectal assembly and an obturator.

### DETAILED DESCRIPTION OF THE INVENTION AND PREFERRED EMBODIMENTS

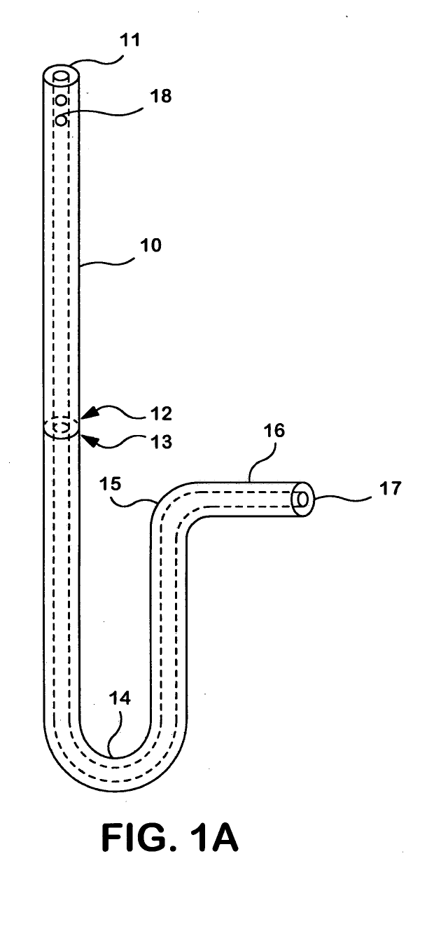

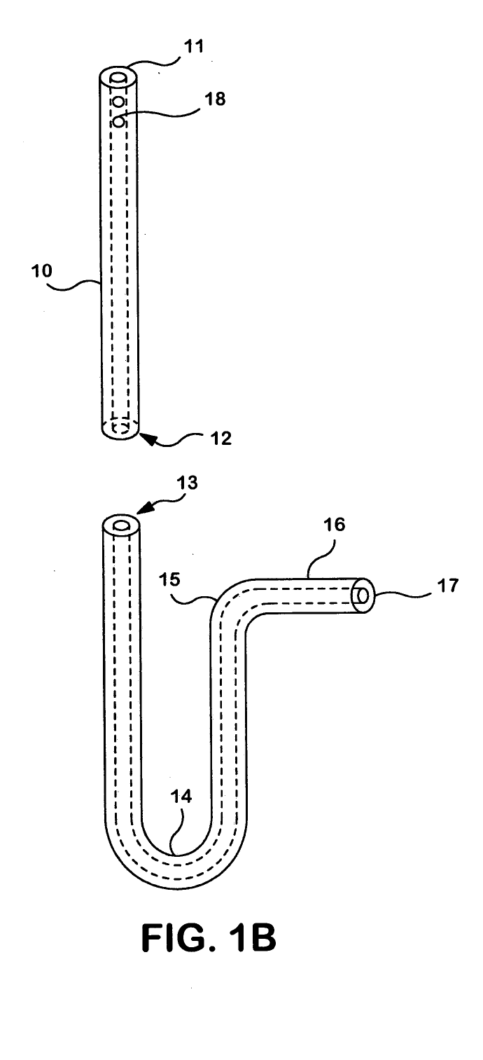

With reference to the drawings there is shown in FIG. 1 a first tubular portion 10 made from a soft pliable material is connected to a curved second tubular portion 14. The first tubular portion 10 has a distal end 11 and a proximal end 12. The second tubular portion 14 also has a distal portion 13 and a proximal end 17. Although not shown, the first tubular portion 10 has a first flexural modulus as measured by ATSM standards wherein the first tubular portion can be inserted, removed and then re-inserted into the rectum without any harm or discomfort to a patient. It will be understood that a suitable flexural modulus for determining what can cause harm or discomfort varies across a range for flexural modulus' but is specifically keyed to preventing injury to the inner colon wall. For example, the flexural modulus is adjusted such that even when inserted improperly into the rectum the inserted portion will not cause colon perforation, sepsis or injury to the anal canal or rectum. While the length of the first tubular portion 10 may vary, the length should not exceed the distance defined between the anus and the sigmoid colon. The diameter of the first tubular portion 10 should also be as small as possible to avoid discomfort yet large enough to allow sufficient fluid flow.

In contrast, the second tubular portion 14 is relatively rigid. The first flex for the first tubular portion 10 is such that the rigidity of the second tubular portion 14 supports the first tubular portion 10 while maintaining a certain minimum bend radius that provides sufficient play to the entire dual flex assembly. The minimum bend radius of the second tubular portion 14 provides a rigid base to the first tubular portion 10 yet enables the user to have more mobility than a hard tube made of rigid plastic or stainless steel.

As shown in FIG. 1A, the second tubular portion 14 can be adapted into a generally “U” or “S” shape as a safety feature or to further add flexibility. The generally “U” or “S” shape is also a means for preventing insertion of the first tubular portion 10 into a user's body cavity beyond a predetermined safe distance. The means may have a variety of shapes such as a double “L” shape, a lateral “U” shape, or a lateral “S” shape in addition to generally “U” and “S” shapes. Notably, the shapes are basically limiters which provide a form of projection or restraint that limits insertion of the first tubular portion 10 too far into the rectum. As the body hits these projections or restraints, the insertion of the first tubular portion 10 into the body is stopped. The projections or restraints are therefore preferably positioned relative to the first tubular portion 10 to stop insertion past a safe distance wherein the safe distance is usually defined by the length of the first tubular portion 10 but can extend beyond such length.

As shown in FIG. 1A, an U-shaped curvature of the second tubular portion 14 forms a curve 15 which prevents insertion of the dual flex assembly too far into the rectum. Other shapes may also be used so long as the shape prevents or provides a reference point to prevent insertion too far past a safe point wherein the means is also sized to prevent easy or unintentionally insertion into a rectum. This means is disposed on the dual flex assembly at a preset distance from the distal end 11. While the previously stated lengths are preferred for colonic application, shorter or longer dimensions may be suitable depending on the specific use of the irrigation apparatus and the person involved.

Also shown in FIG. 1A is a proximal end 17 of the second tubular portion 14. The proximal end 17 can be adapted to receive fluid from any fluid delivery means where a preferred embodiment of the invention contemplates a Sitolonic™ toilet attachment. As described in U.S. Pat. No. 5,946,741, the disclosure of which is incorporated herein by reference, the Sitolonic™ is a toilet attachment configured to be positioned between a toilet seat and a toilet bowl. In use, the Sitolonic™ toilet attachment is secured by the weight of the toilet seat wherein the Sitolonic™ toilet attachment has a base plate and a tubular irrigation assembly which can be connected to a fluid line. The base plate has an upper and lower surface, a rear end and a front end. When the base plate is installed, the rear end faces a rear portion of the bowl and the front end faces a front portion of the bowl. The base plate also has ports to receive and secure the fluid line and the irrigation assembly. The irrigation assembly of the Sitolonic™ device comprises an irrigation tip, and in one embodiment of the toilet attachment, has a limiter to prevent the insertion of the tip too far into a body cavity.

As it relates to the present invention, the irrigation tip of the Sitolonic™ toilet attachment can be configured to be in mutual communication with the second tubular portion 14. Alternatively, the irrigation tip of the Sitolonic™ can be replaced by the dual flex assembly device of the invention. Means such as a barb, screw or clamp can be used to secure the distal end 17 of the second tubular portion 14 to the fluid line of the Sitolonic™. A horizontal portion 16 of second tubular portion 14 can be used to aid in attachment or removal of the dual flex assembly to the Sitolonic™ toilet attachment. However, it should be noted that the invention is not limited to the Sitolonic™ and can be used in conjunction with any other type of colonic or irrigation devices as well as other toilet attachments known within the art wherein the distal end 17 can be placed in communication with any number of fluid lines.

Notably, the proximal end 12 of the first tubular portion 10 can be removably attached or mutually welded to the distal end 13 of the second tubular portion 14. As shown in FIG. 1A, the proximal end 12 and distal end 13 can be cut flat and attached by any means including glue, welding or any other mechanical means. FIG. 1B shows an exploded view of the first tubular portion 10 and a second tubular portion 14 prior to welding. Alternatively, the manufacturing process can be designed to form separate flexural moduli during fabrication wherein the mechanical means for joining the proximal end 12 and distal end 13 are not required.

Also shown in FIG. 1A in an area adjacent to the distal end 11 is a plurality of apertures 18 disposed on the first tubular portion 11. The apertures 18 allow for easy flow of fluid into the rectum. The apertures 18 can be placed along the length of the first tubular portion 10 to maximize saturation of the colon. Typically, the apertures 18 may have a diameter of about 3/32 inch and are uniformly distributed over the area adjacent to the distal end 11. The distal end 11 may also have an enlarged bulbous shape suitable for a douche.

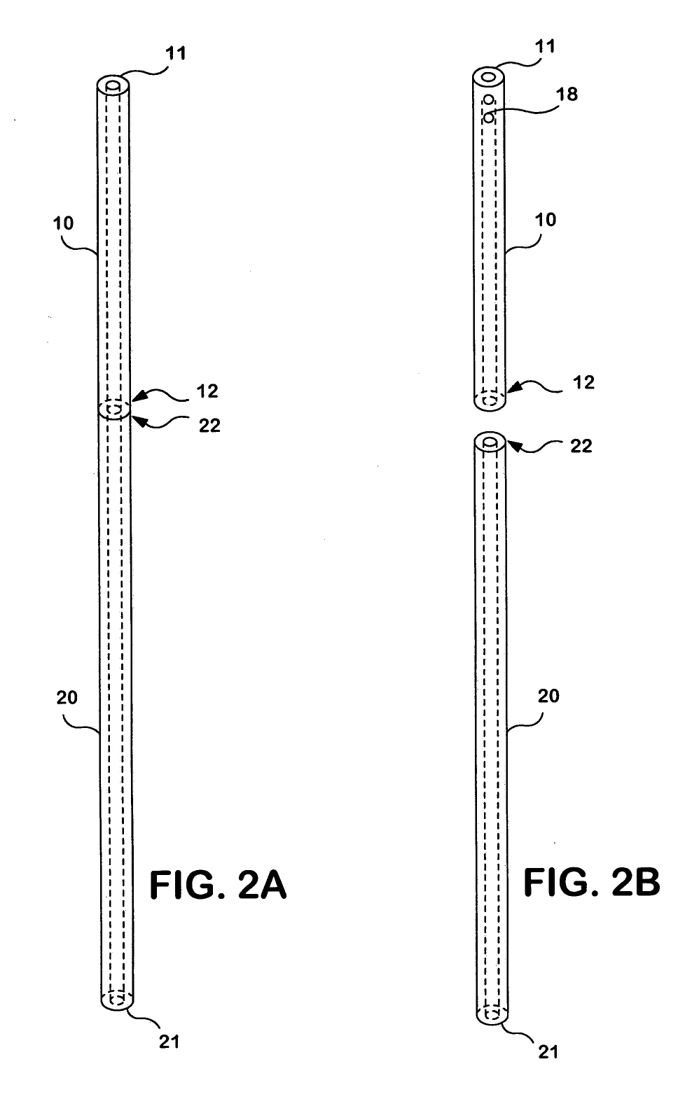

Typically, the length of the first tubular portion 10 is the length desired to be inserted into the rectum. In one embodiment, the first tubular portion 10 is 3 inches and has an outer diameter of ¼ inch and an inner diameter of ⅛ inch. However, as shown in FIGS. 2A and 2B, the length of the second tubular portion 14 can be adapted to any number of various shapes or lengths. For example, the second tubular portion 20 of FIG. 2A has a straight length of 9 inches long in contrast to the generally shaped “U” tubular portions of FIGS. 1A and 1B. The second tubular portion 20 has a distal end 22 and a proximal end 21 wherein the distal end 22 is in communication with the proximal end 22 of the first tubular portion 10. These embodiments can be preferentially used in conjunction with Colemna Boards® or any other flat surface used in connection with enemas for patients lying in a supine position.

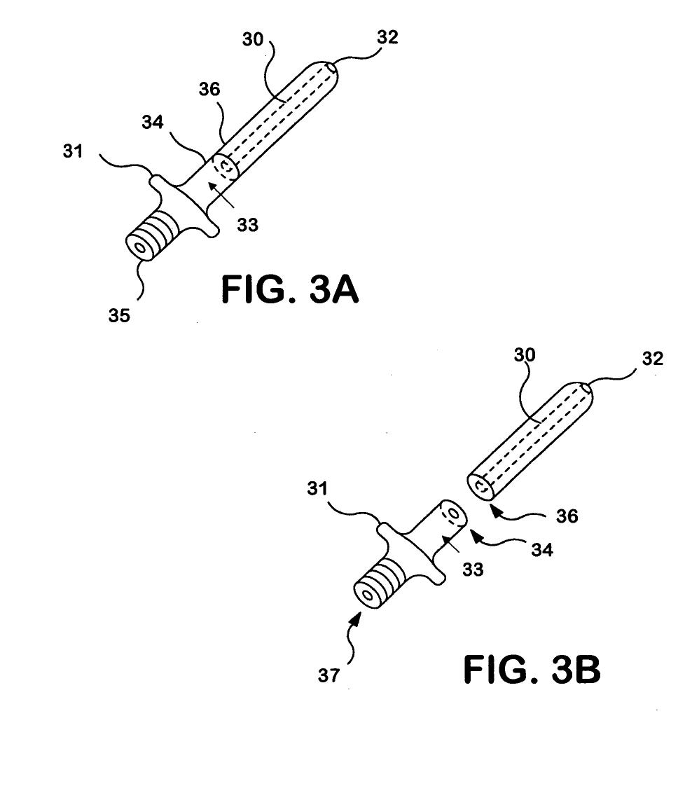

FIG. 3A shows an embodiment where the second tubular portion 33 can be shaped into a standard enema tip for use with any number of enema devices, colonics, irrigators, or toilet attachments. The first tubular portion 30 while being similar in composition, length, diameter and flexural modulus to the first tubular portion 10, can also be shaped to have a round end instead of being cut flat on the distal end 32 of the first tubular portion 30. Moreover, the second tubular portion 33 can be shaped to have flanges 31 disposed on the circumference of the second tubular portion 33 at such a point to prevent insertion of the dual flex assembly into the rectum past a safe point. These flanges 31 are similar to the limiting or restricting means as shown in FIGS. 1A and 2A. The second tubular portion 33 can also be barbed in an area adjacent to the proximal end 35. The proximal end 36 of the first tubular potion 30 can also be cut flat to be in communication with a distal end 34 of the first tubular portion 33. Furthermore, and as shown in FIG. 3B, the proximal end 35 of the second tubular portion 33 can be adapted to receive fluid in a direction 37.

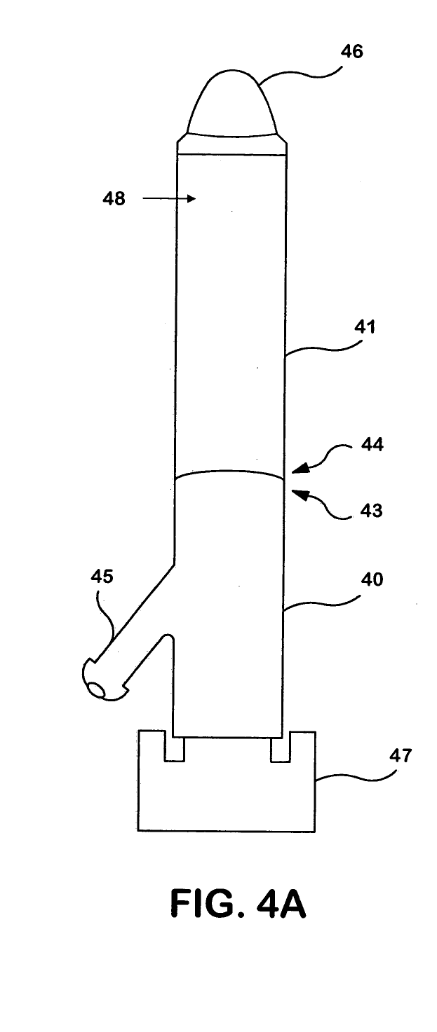

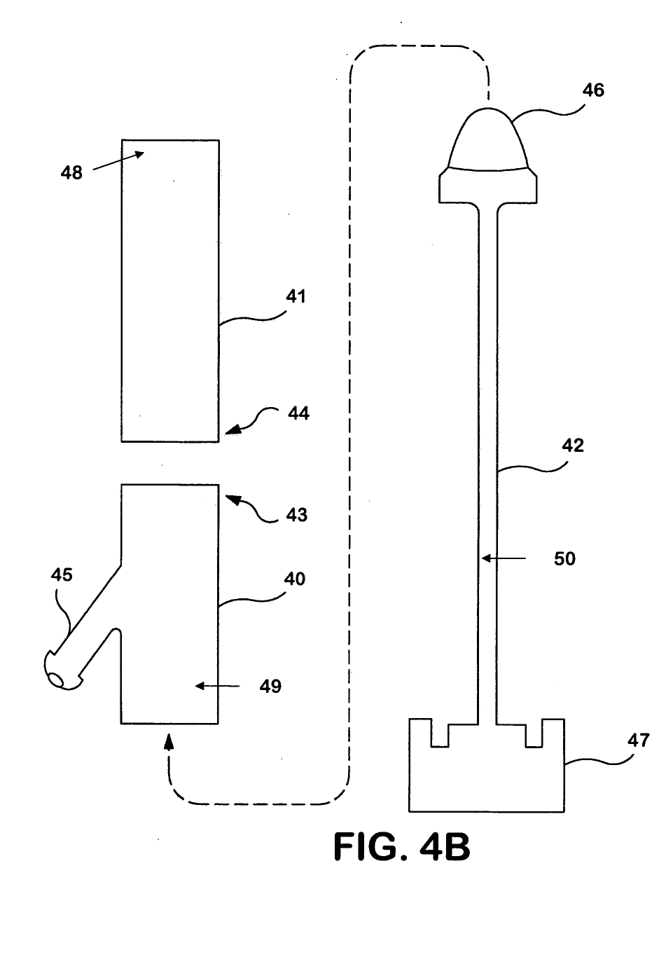

FIG. 4A shows a dual flex assembly adapted for use with an obturator or a speculum as shown in U.S. Pat. No. 4,842,580, the disclosure of which is incorporated herein by reference. As shown in FIG. 4A, the first tubular portion 41 is in communication with a second tubular portion 40 wherein a proximal portion 44 of the first tubular portion 41 is provided as well as a distal portion 43 of the second tubular portion 40. Both the proximal portion 44 of the first tubular portion 41 and the distal portion 43 of the second tubular portion 40 is in communication with each other according to any of the methods provided herein. However, the distal portion 48 of the first tubular portion 41 does not first penetrate the rectum in the present embodiment. Instead, an insertion tip 46 on the end of an obturator or speculum first penetrates the rectum rather than a distal portion 48 of the first tubular portion 41.

As shown in FIG. 4A, the proximal end 49 of second tubular portion 40 sits in an end plate 47 adapted to receive the proximal end 49 of the second tubular portion 40 wherein the end plate 47 is affixed to a shaft 42 of an obturator as shown in FIG. 4B. In particular, the first tubular portion 41 has the same first flexural modulus to prevent traumatic injury as discussed herein. The distal end 48 can also be optionally tapered which is not shown in the FIGS. 4A and 4B to allow a rounded head 46 to project from the distal end 48 of the first tubular portion 41. In particular, the obturator 50 has a rounded nose 46 attached to a shaft 42 and a base 47. Upon insertion of the obturator into the first and second tubular portion as shown in FIG. 4B, the rounded-nose head 46 is in a direction towards the distal end 48 of the first tubular portion 41 wherein the rounded head 46 protrudes from the distal end 48. This relation provides a smooth transition between the two parts and results in painless entry of the first tubular portion 41 into the rectum. After insertion into the rectum, the obturator bearing against the inner surface of the tapered open end may be withdrawn from the dual flex assembly.

In one embodiment of the invention, the shaft 42 or alternatively the dual flex assembly may be slightly offset in relation to the longitudinal axis of the dual flex assembly device when straightened to result in fluid entering the dual flex assembly device though a nozzle 45 adapted to the second tubular section. The fluid is directed to one side and adopts a spiral, or swirling, path.

The dual flex assembly of the present invention has been described with reference to particular embodiments and exemplifications thereof. It is to be understood that the present invention may be employed in combination with toilet attachment and rectal tips of designs other than those depicted herein. Variation in design of some of the elements thereof may occur to one skilled in the art without departing from the scope of the present invention. The scope of the present invention is not intended to be limited to the embodiments and exemplifications depicted herein, but solely by the claims appended hereto and all equivalents thereof.

1. A dual flex assembly device for delivering liquid into the rectum, comprising:
a first tubular portion having a proximal end, a distal functional end, and a first flexural modulus wherein said first flexural modulus is sufficiently pliable to allow for repeated non-traumatic insertion and removal of the first tubular portion into and from the rectum,
said proximal end of the first tubular portion being in communication with a second tubular portion also having a proximal end, a distal end, and a second flexural modulus wherein said second flexural modulus is sufficiently rigid to support the first tubular portion but also has a minimum bend radius that allows for sufficient play of the second tubular portion that preferentially allows for insertion of the distal end of the first tubular portion into the rectum
said proximal end of the second tubular portion being in communication with a means for delivering fluid into the second tubular portion and thereby into the rectum.

2. The dual flex assembly device of claim 1 wherein said proximal portion of said first tubular portion is removably attached to said distal end of said second tubular portion.

3. The dual flex assembly device of claim 1 wherein said proximal end of said first tubular portion is mutually welded to said distal end of second tubular portion.

4. The dual flex assembly device of claim 1 wherein said second tubular portion has a generally “U” shape.

5. The dual flex assembly device of claim 1 wherein said distal portion of said first tubular portion is generally adapted to allow for easy insertion of the distal end into the rectum and having apertures disposed on said distal end of said first tubular portion.

6. The dual flex assembly device of claim 1 wherein said proximal end of said second tubular portion is generally adapted to a toilet attachment.

7. The dual flex assembly device of claim 6 wherein said toilet attachment is positioned between a toilet seat and a toilet bowl and is connected to a fluid line, comprising:
a tubular irrigation assembly comprising a U-shaped tube having an irrigation tip defining an outlet for fluid received from the fluid line at a first end, a U-shaped portion having an inlet for receiving fluid from the fluid line at an opposite second end thereof, and a limiter disposed between said irrigation tip and said U-shaped portion at a preset distance from the outlet to limit the distance the tip is inserted into a body cavity during use and for increasing the flexibility of the insertion tip, the limiter being located along a plane passing through a longitudinal axis of the irrigation assembly; and
a base plate, said base plate having an upper and lower surface, a rear end and a front end, and first and second ports in the base plate, one to receive and secure the fluid line and one to receive and secure the irrigation assembly to provide fluid for passage through the irrigation assembly, at least one of said ports being located at said front end, said base plate having at least a portion sized to fit between the toilet seat and the toilet bowl when the seat rests against the bowl.

8. The dual flex assembly device of claim 1, further comprising:
an insertion limiter extending laterally from the second tubular portion at a preset distance from the distal end of said first tubular portion to limit insertion of the first tubular portion into a user's body.

9. The dual flex assembly device of claim 1 wherein said second tubular portion has a generally double “U” shape.

10. The dual flex assembly device of claim 1 where said first tubular portion and second tubular portion are comprised of a material selected from the group consisting of synthetic polymer, polyvinyl chloride, polyethylene, polypropylene, polybutadiene, polyester, nylon, and the like and blends and copolymers thereof.

11. The dual flex assembly device of claim 1 wherein said distal end of said first tubular portion is 3 inches long and has a ¼ inch outer diameter and a ⅛ inch inner diameter.

12. The dual flex assembly device of claim 1 wherein said proximal end of second tubular portion is barbed and said distal end is flat and mutually welded to said proximal end of said first tubular portion.

13. The dual flex assembly device of claim 1 wherein said first and second tubular portions are adapted for receiving an obturator wherein said obturator is comprised of a bulbous end and a second end separated by a shaft, said bulbous end is being oriented with the distal end of said first tubular portion and being adapted to fit into a patient's rectum wherein said shaft has a third flexural modulus which is at least as flexible as said first flexural modulus of said first tubular part.

14. The dual flex assembly device of claim 13 wherein a water hose barb is disposed on said second tubular portion.

15. The dual flex assembly device of claim 13 wherein said second end of the obturator is adapted to receive the proximal end of the second tubular portion and is removably affixed thereto.

16. The dual flex assembly device of claim 13 wherein said first and second tubular portion have an outer diameter of 1 inch.

17. The dual flex assembly device of claim 13 wherein said water hose barb is disposed on said second tubular portion.
